(19) United States
(12) Patent Application Publication
(10) Pub. No.: US 2026/0294991 A1
(43) Pub. Date: Oct. 1, 2026

(54) Use of Deep Eutectic Solvent Extract of Chinese Medicinal Herb Composition

(71) Applicants: Chloex Biotech Co., Ltd., Chiayi City (TW); Nanosigma Biotech. Co., Ltd., New Taipei City (TW)

(72) Inventors: Cheng-Nan Chen, Chiayi City (TW); Chih-Hsien Lin, New Taipei City (TW)

(21) Appl. No.: 19/097,084

(22) Filed: Apr. 1, 2025

(51) Int. Cl. A61K 36/282; A61K 9/06; A61K 36/355; A61K 36/534; A61K 36/61; A61K 36/748; A61K 36/78; A61K 47/02; A61K 47/12; A61K 47/18; A61K 47/26; A61K 47/32; A61K 47/38; A61P 17/10; A61P 29/00; A61P 31/04

(52) U.S. Cl. CPC A61K 36/282; A61K 9/06; A61K 36/355; A61K 36/534; A61K 36/61; A61K 36/748; A61K 36/78; A61K 47/02; A61K 47/12; A61K 47/183; A61K 47/26; A61K 47/32; A61K 47/38; A61P 17/10; A61P 29/00; A61P 31/04; A61K 2236/333; A61K 2236/51

(57) ABSTRACT

A use of a deep eutectic solvent extract of a Chinese medicinal herb composition in preparing a pharmaceutical composition with antibacterial activity is provided. The extract is obtained by performing ultrasonic extraction on the Chinese medicinal herb composition, which is composed of Artemisia argyi, Houttuynia cordata, Hedyotis diffusa, Anisomeles indica (L.) Kuntze, and Lonicera japonica, through a deep eutectic solvent and by performing a subsequent concentration process. Using a deep eutectic solvent that meets the requirements of green chemistry not only allows the active ingredients of the Chinese medicinal herb composition to be effectively extracted for use in preparing a pharmaceutical composition with antibacterial and anti-inflammatory activity, but also meets the green chemistry requirements by eliminating the need to use an organic solvent. The deep eutectic solvent is also advantageous in that it does not evaporate, has low toxicity, is biodegradable, renewable, and inexpensive, and can be easily produced.

### BACKGROUND

### Field of the Invention

The present invention relates to the extraction and application of the active ingredients of a Chinese medicinal herb composition. More particularly, the invention relates to a use of a deep eutectic solvent extract of a Chinese medicinal herb composition, wherein the deep eutectic solvent extract is obtained by extracting the active ingredients of the Chinese medicinal herb composition with a deep eutectic solvent.

### Description of Related Art

Chinese medicinal herbs are rich in bioactive ingredients and therefore have great value in medical use, with applications covering disease prevention, disease treatment, and health management. However, the extraction and use of Chinese medicinal herb ingredients still face multiple challenges, including how to extract the active ingredients in a highly effective and eco-friendly manner. The choice of the extraction technique and solvent is critical to the extraction rate, extraction quality, and the effect of the intended medical use.

The conventional extraction methods generally rely on organic solvents such as ethanol or chloroform. These solvents advantageously feature high efficiency but are disadvantaged by their high toxicity, volatility, and grave impact on the environment. Besides, the recycling and treatment of organic solvents consume a large amount of energy, and this not only increases production cost, but also poses a challenge to sustainable development. Recently, therefore, green extraction techniques have attracted much attention. Green extraction aims to use eco-friendly, renewable, and low-toxicity solvents to reduce energy consumption, to promote the recycling and reuse of waste, and to thereby create a win-win situation in terms of economic benefits and environmental protection.

Natural deep eutectic solvents (NaDESs) are innovative solvents for use in green extraction and have significant advantages. A NaDES is a deep eutectic mixture of two or more primary metabolites (e.g., sugars, organic acids, and amino acids) in a specific mole ratio and works on the principle of hydrogen bonding to achieve a low melting point, renewability, low toxicity, and biodegradability, among other features. Compared with the traditional organic solvents, NaDESs not only can increase the extraction efficiency of the active ingredients of plants significantly, but also can ensure the stability and storability of the extracted compounds. Thanks to their polarity tunability and high dissolving power over different hydrophilic or hydrophobic compounds, NaDESs have become a paradigm of “design solvents.”

Studies have shown that the ingredients of a NaDES and their ratio have direct impact on the extraction effect. In particular, the physical and chemical properties of a NaDES, such as polarity, viscosity, and dissolving power, play a critical role in the extraction process. NaDESs have been successfully used in the extraction of active ingredients in the fields of cosmetics, pharmaceutics, and food. For example, NaDESs can be used to extract phenol-based compounds from plants with high efficiency and have been proved to have great application potential in sunscreens, food preservatives, protein stabilizers, and so on.

While NaDESs have shown tremendous application potential in various fields, research on their use in skincare, medical materials, and pharmaceutics is still limited, comparatively speaking. Further exploration of the application of NaDESs in the aforesaid fields not only contributes to sustainable development of the related industries, but also may provide new technical solutions to highly efficient use of Chinese medicinal herbs.

### SUMMARY

To overcome the technical problem stated above, it is an objective of the present invention to provide a use of a deep eutectic solvent extract of a Chinese medicinal herb composition. The extract is obtained by performing ultrasonic extraction on the Chinese medicinal herb composition (which is composed of Artemisia argyi, Houttuynia cordata, Hedyotis diffusa, Anisomeles indica (L.) Kuntze, and Lonicera japonica) using a deep eutectic solvent that meets the requirements of green chemistry. The deep eutectic solvent not only allows the active ingredients of the Chinese medicinal herb composition to be effectively extracted for use in preparing a pharmaceutical composition with antibacterial activity, but also meets the green chemistry requirements by eliminating the need to use an organic solvent. The deep eutectic solvent is also advantageous in that it does not evaporate, has low toxicity, is biodegradable, renewable, and inexpensive, and can be easily produced.

To achieve the foregoing objective, the present invention provides a use of a deep eutectic solvent extract of a Chinese medicinal herb composition in preparing a pharmaceutical composition with antibacterial and anti-inflammatory activity. The extract is obtained by performing ultrasonic extraction on the Chinese medicinal herb composition through a deep eutectic solvent and by subsequently performing a concentration process. The hydrogen bond acceptor (HBA) of the deep eutectic solvent is betaine (Bet), and the hydrogen bond donor (HBD) of the deep eutectic solvent is selected from the group consisting of glycerol (Gly), lactate (LA), and mannose (MAN). The Chinese medicinal herb composition is selected from the group consisting of a combination of Artemisia argyi, Houttuynia cordata, Hedyotis diffusa, Anisomeles indica (L.) Kuntze, and Lonicera japonica and a combination of Mentha spp, and Melaleuca alternifolia.

In one embodiment of the present invention, the ingredients of the Chinese medicinal herb composition include: Artemisia argyi, Houttuynia cordata, Hedyotis diffusa, and Anisomeles indica (L.) Kuntze, the entire plants (including leaves, stalks/stems, and roots) of the four of which can be used as the medicinal portions; and Lonicera japonica, the flowers, leaves, and stalks/stems of which can be used as the medicinal portions. The medicinal portions of the aforesaid ingredients of the Chinese medicinal herb composition are subjected to room-temperature/hot-air drying at 40° C. to 60° C. and then mixed with a deep eutectic solvent to form the raw material for ultrasonic extraction. Preferably, the dried Chinese medicinal herb composition is further ground to form powdery particles that can pass through a sieve of mesh size 20 to 60, i.e., to form a powdery Chinese medicinal herb composition, in order to increase the efficiency of mixing with the deep eutectic solvent in preparation for ultrasonic extraction.

In one embodiment of the present invention, the ingredients of the Chinese medicinal herb composition include: Mentha spp., the entire plant (including leaves, stalks/stems, and roots) of which can be used as the medicinal portion; and Melaleuca alternifolia, the leaves of which can be used as the medicinal portion. The medicinal portions of the aforesaid ingredients of the Chinese medicinal herb composition are subjected to room-temperature/hot-air drying at 40° C. to 60° C. and then mixed with a deep eutectic solvent to form the raw material for ultrasonic extraction. Preferably, the dried Chinese medicinal herb composition is further ground to form powdery particles that can pass through a sieve of mesh size 20 to 60, i.e., to form a powdery Chinese medicinal herb composition, in order to increase the efficiency of ultrasonic extraction performed after mixing the Chinese medicinal herb composition with the deep eutectic solvent.

In one mode of implementation of the present invention, the deep eutectic solvent may use betaine (abbreviated herein as Bet, with the molecular formula C₅H₁₁NO₂) as the hydrogen bond acceptor and use glycerol (abbreviated herein as Gly, with the molecular formula C₃H₈O₃), lactate (abbreviated herein as LA, with the molecular formula C₁₂H₂₂O₁₁), or mannose (abbreviated herein as MAN, with the molecular formula C₆H₁₂O₆) as the hydrogen bond donor. In one mode of implementation, the mole ratio between the hydrogen bond acceptor and the hydrogen bond donor of the deep eutectic solvent may be in the range from 3:1 to 1:3. In another mode of implementation, the hydrogen bond acceptor and the hydrogen bond donor of the deep eutectic solvent may be in a mole ratio of 3:1, 2:1, 1:0.5, 1:1, 1:1.5, 1:2, 1:2.5, or 1:3, or the mole ratio between the hydrogen bond acceptor and the hydrogen bond donor of the deep eutectic solvent may be in a range defined by, for example but not limited to, any two of the foregoing mole ratios.

In one mode of implementation of the present invention, the deep eutectic solvent may use betaine (Bet) as the hydrogen bond acceptor and use glycerol (Gly), lactate (LA), or mannose (MAN) as the hydrogen bond donor. In one mode of implementation, the deep eutectic solvent is prepared by mixing betaine and glycerol in a mole ratio in the range from 1:1.5 to 1:2.5, and preferably, the mole ratio of betaine to glycerol is 1:2. In another mode of implementation, the deep eutectic solvent is prepared by mixing betaine and lactate in a mole ratio in the range from 1:0.5 to 1:1.5, and preferably, the mole ratio of betaine to lactate is 1:1. In yet another mode of implementation, the deep eutectic solvent is prepared by mixing betaine and mannose in a mole ratio in the range from 1:0.5 to 1:1.5, and preferably, the mole ratio of betaine to mannose is 1:1.

In one mode of implementation of the present invention, the Chinese medicinal herb composition and the deep eutectic solvent may be in a solid-to-liquid weight ratio in the range from 1:5 to 1:15. In one mode of implementation, the solid-to-liquid weight ratio between the Chinese medicinal herb composition and the deep eutectic solvent may be 1:3, 1:6, 1:9, 1:10, 1:13, or 1:15, or the solid-to-liquid weight ratio between the Chinese medicinal herb composition and the deep eutectic solvent may be in a range defined by, for example but not limited to, any two of the foregoing ratios. Preferably, the solid-to-liquid weight ratio between the Chinese medicinal herb composition and the deep eutectic solvent is 1:10.

In one mode of implementation of the present invention, the ultrasonic extraction may be carried out using the ultrasonic extraction system and equipment of Taiwan Patent No. 1783508B so as to achieve the objective of preserving a relatively large amount of thermosensitive plant-based chemical substances through low-temperature extraction.

In one mode of implementation of the present invention, the ultrasonic extraction is performed at an extraction temperature in the range from 25° C. to 35° C. Preferably, the extraction temperature is 25° C., 26° C., 27° C., 28° C., 29° C., 30° C., 31° C., 32° C., 33° C., 34° C., or 35° C., or the extraction temperature may be in a range defined by, for example but not limited to, any two of the foregoing values.

In one mode of implementation of the present invention, the ultrasonic extraction lasts for an extraction time in the range from 30 minutes to 60 minutes. Preferably, the extraction time is 30 minutes, 35 minutes, 40 minutes, 45 minutes, 50 minutes, 55 minutes, or 60 minutes, or the extraction time may be in a range defined by, for example but not limited to, any two of the foregoing values.

In one mode of implementation of the present invention, the ultrasonic extraction is performed at an ultrasonic power in the range from 1000 W to 3000 W and at an ultrasonic frequency in the range from 20 KHz to 100 KHz. Preferably, the ultrasonic power is 1000 W, 1200 W, 1400 W, 1600 W, 1800 W, 2000 W, 2200 W, 2400 W, 2600 W, 2800 W, or 3000 W, or the ultrasonic power may be in a range defined by, for example but not limited to, any two of the foregoing values. Preferably, the ultrasonic frequency is 20 KHz, 30 kHz, 40 KHz, 50 kHz, 60 kHz, 70 KHz, 80 kHz, 90 kHz, or 100 kHz, or the ultrasonic frequency may be in a range defined by, for example but not limited to, any two of the foregoing values.

In one mode of implementation of the present invention, the pharmaceutical composition may be a pharmaceutical preparation or a liquid gel dressing. When the pharmaceutical composition is a liquid gel dressing, the deep eutectic solvent extract obtained from the Chinese medicinal herb composition by extraction with the deep eutectic solvent may be further dissolved or dispersed in a carrier solvent together with another adjuvant such that the deep eutectic solvent extract, the adjuvant, and the carrier solvent are mixed together and jointly form the liquid gel dressing. More specifically, the carrier solvent may be water or a herbal distillate, wherein the water may be selected from the group consisting of purified water, water for injection (WFI), distilled water, sterile water, and electrolyzed reduced water, and wherein the herbal distillate may be a Melaleuca alternifolia distillate.

In one mode of implementation of the present invention, with the total weight of the Chinese medicinal herb composition constituting 100 wt %, the Chinese medicinal herb composition may include 20 wt % to 25 wt % of Artemisia argyi, 15 wt % to 20 wt % of Houttuynia cordata, 15 wt % to 20 wt % of Hedyotis diffusa, 9 wt % to 13 wt % of Anisomeles indica (L.) Kuntze, and 27 wt % to 32 wt % of Lonicera japonica. More specifically, with the total weight of the Chinese medicinal herb composition constituting 100 wt %, Artemisia argyi may make up, for example but not limited to, 20 wt %, 21 wt %, 22 wt %, 23 wt %, 24 wt %, or 25 wt %, or each of the foregoing values may serve as an endpoint value of the range of the weight percent of Artemisia argyi, Houttuynia cordata may make up, for example but not limited to, 15 wt %, 16 wt %, 17 wt %, 18 wt %, 19 wt %, or 20 wt %, or each of the foregoing values may serve as an endpoint value of the range of the weight percent of Houttuynia cordata; Hedyotis diffusa may make up, for example but not limited to, 15 wt %, 16 wt %, 17 wt %, 18 wt %, 19 wt %, or 20 wt %, or each of the foregoing values may serve as an endpoint value of the range of the weight percent of Hedyotis diffusa; Anisomeles indica (L.) Kuntze may make up, for example but not limited to, 9 wt %, 10 wt %, 11 wt %, 12 wt %, or 13 wt %, or each of the foregoing values may serve as an endpoint value of the range of the weight percent of Anisomeles indica (L.) Kuntze; and Lonicera japonica may make up, for example but not limited to, 27 wt %, 28 wt %, 29 wt %, 30 wt %, 31 wt %, or 32 wt %, or each of the foregoing values may serve as an endpoint value of the range of the weight percent of Lonicera japonica.

Preferably, with the total weight of the Chinese medicinal herb composition constituting 100 wt %, the Chinese medicinal herb composition includes 22.7 wt % of Artemisia argyi, 18.2 wt % of Houttuynia cordata, 18.2 wt % of Hedyotis diffusa, 11.4 wt % of Anisomeles indica (L.) Kuntze, and 29.5 wt % of Lonicera japonica.

In one mode of implementation of the present invention, the pharmaceutical composition includes a liquid gel dressing, and with the liquid gel dressing constituting 100 wt %, the liquid gel dressing includes 10 wt % to 20 wt % of the deep eutectic solvent extract, 5 wt % to 11 wt % of polyvinyl pyrrolidone (PVP), 1 wt % to 3 wt % of hydroxypropyl methylcellulose (HPMC), 0.1 wt % to 0.3 wt % of calcium chloride (CaCl₂)), 0.05 wt % to 0.15 wt % of disodium ethylenediaminetetraacetate (EDTA-2Na), and water making up the remaining percentage. More specifically, with the liquid gel dressing constituting 100 wt %, the deep eutectic solvent extract may make up, for example but not limited to, 10 wt %, 11 wt %, 12 wt %, 13 wt %, 14 wt %, 15 wt %, 16 wt %, 17 wt %, 18 wt %, 19 wt %, or 20 wt %, or each of the foregoing values may serve as an endpoint value of the range of the weight percent of the deep eutectic solvent extract; PVP may make up, for example but not limited to, 5 wt %, 6 wt %, 7 wt %, 8 wt %, 9 wt %, 10 wt %, or 11 wt %, or each of the foregoing values may serve as an endpoint value of the range of the weight percent of PVP; HPMC may make up, for example but not limited to, 1 wt %, 1.5 wt %, 2 wt %, 2.5 wt %, or 3 wt %, or each of the foregoing values may serve as an endpoint value of the range of the weight percent of HPMC; CaCl₂) may make up, for example but not limited to, 0.1 wt %, 0.15 wt %, 0.2 wt %, 0.25 wt %, or 0.3 wt %, or each of the foregoing values may serve as an endpoint value of the range of the weight percent of CaCl₂); and EDTA-2Na may make up, for example but not limited to, 0.05 wt %, 0.1 wt %, or 0.15 wt %, or each of the foregoing values may serve as an endpoint value of the range of the weight percent of EDTA-2Na.

Preferably, the liquid gel dressing includes 15 wt % of the deep eutectic solvent extract, 8 wt % of PVP, 2 wt % of HPMC, 0.2 wt % of CaCl₂), 0.1 wt % of EDTA-2Na, and water making up the remaining percentage.

In one mode of implementation of the present invention, with the Chinese medicinal herb composition including Artemisia argyi, Houttuynia cordata, Hedyotis diffusa, Anisomeles indica (L.) Kuntze, and Lonicera japonica, the pharmaceutical composition includes the aforesaid liquid gel dressing and water additionally added to the liquid gel dressing, and with the liquid gel dressing constituting 100 wt %, the additionally added water may make up 20 wt % to 70 wt %. More specifically, the additionally added water may make up, for example but not limited to, 20 wt %, 25 wt %, 30 wt %, 35 wt %, 40 wt %, 45 wt %, 50 wt %, 55 wt %, 60 wt %, 65 wt %, or 70 wt %, or each of the foregoing values may serve as an endpoint value of the range of the weight percent of the additionally added water.

In one mode of implementation of the present invention, with the total weight of the Chinese medicinal herb composition constituting 100 wt %, the Chinese medicinal herb composition may include 45 wt % to 55 wt % of Mentha spp, and 45 wt % to 55 wt % of Melaleuca alternifolia. More specifically, with the total weight of the Chinese medicinal herb composition constituting 100 wt %, Mentha spp. may make up, for example but not limited to, 45 wt %, 47 wt %, 49 wt %, 50 wt %, 51 wt %, 53 wt %, or 55 wt %, or each of the foregoing values may serve as an endpoint value of the range of the weight percent of Mentha spp.; and Melaleuca alternifolia may make up, for example but not limited to, 45 wt %, 47 wt %, 49 wt %, 50 wt %, 51 wt %, 53 wt %, or 55 wt %, or each of the foregoing values may serve as an endpoint value of the range of the weight percent of Melaleuca alternifolia.

Preferably, with the total weight of the Chinese medicinal herb composition constituting 100 wt %, the Chinese medicinal herb composition includes 50 wt % of Mentha spp, and 50 wt % of Melaleuca alternifolia.

In one mode of implementation of the present invention, with the Chinese medicinal herb composition including Mentha spp, and Melaleuca alternifolia, the pharmaceutical composition including a liquid gel dressing, and the liquid gel dressing constituting 100 wt %, the liquid gel dressing includes 10 wt % to 20 wt % of the deep eutectic solvent extract, 5 wt % to 11 wt % of PVP, 1 wt % to 3 wt % of HPMC, and a Melaleuca alternifolia distillate making up the remaining percentage. More specifically, with the liquid gel dressing constituting 100 wt %, the deep eutectic solvent extract may make up, for example but not limited to, 10 wt %, 11 wt %, 12 wt %, 13 wt %, 14 wt %, 15 wt %, 16 wt %, 17 wt %, 18 wt %, 19 wt %, or 20 wt %, or each of the foregoing values may serve as an endpoint value of the range of the weight percent of the deep eutectic solvent extract; PVP may make up, for example but not limited to, 5 wt %, 6 wt %, 7 wt %, 8 wt %, 9 wt %, 10 wt %, or 11 wt % or each of the foregoing values may serve as an endpoint value of the range of the weight percent of PVP; and HPMC may make up, for example but not limited to, 1 wt %, 1.5 wt %, 2 wt %, 2.5 wt %, or 3 wt %, or each of the foregoing values may serve as an endpoint value of the range of the weight percent of HPMC.

Preferably, the liquid gel dressing includes 15 wt % of the deep eutectic solvent extract, 8 wt % of PVP, 2 wt % of HPMC, and a Melaleuca alternifolia distillate making up the remaining percentage.

In one mode of implementation of the present invention, the pharmaceutical composition is a liquid gel dressing for inhibiting the growth activity of Cutibacterium acnes (C. acnes).

In one mode of implementation of the present invention, with the deep eutectic solvent being a combination of betaine and mannose (Bet-MAN), the liquid gel dressing is additionally added with water making up 55 wt % to 65 wt % of the liquid gel dressing to form a first diluted gel. The first diluted gel can be used to inhibit the growth activity of C. acnes, Staphylococcus epidermidis (S. epidermidis), Staphylococcus aureus (S. aureus), or Pseudomonas aeruginosa (P. aeruginosa).

In one mode of implementation of the present invention, the first diluted gel can be used to inhibit inflammation and does not have cytotoxicity toward keratinocytes (HaCaT cells) or THP-1 macrophages.

In one mode of implementation of the present invention, the first diluted gel can be used to inhibit C. acnes-induced IL-1B expression and C. acnes-induced IL-8 expression in keratinocytes (HaCaT cells).

In one mode of implementation of the present invention, the first diluted gel can be used to inhibit C. acnes-induced IL-1B expression and C. acnes-induced IL-8 expression in THP-1 macrophages.

In one mode of implementation of the present invention, with the deep eutectic solvent being a combination of betaine and lactate (Bet-LA), the liquid gel dressing is additionally added with water making up 30 wt % to 40 wt % of the liquid gel dressing to form a second diluted gel. The second diluted gel can be used to inhibit the growth activity of C. acnes, S. epidermidis, S. aureus, or P. aeruginosa.

The techniques and means adopted by the present invention to achieve the aforesaid objectives and other effects are detailed below with reference to some preferred feasible embodiments of the invention.

### BRIEF DESCRIPTION OF THE DRAWINGS

FIG. 1A shows the results of the antibacterial activity test of the liquid gel dressings according to Embodiments 1A of the present invention in Test Example 1;

FIG. 1B shows the results of the antibacterial activity test of the liquid gel dressings according to Embodiments 2A of the present invention in Test Example 1;

FIG. 1C shows the results of the antibacterial activity test of the liquid gel dressings according to Embodiments 3A of the present invention in Test Example 1;

FIG. 2A shows the results of the antibacterial activity test against C. acnes in Test Example 2 of the diluted gel dressings according to Embodiments 1B and 2B of the present invention;

FIG. 2B shows the results of the antibacterial activity test against S. epidermidis in Test Example 2 of the diluted gel dressings according to Embodiments 1B and 2B of the present invention;

FIG. 2C shows the results of the antibacterial activity test against S. aureus in Test Example 2 of the diluted gel dressings according to Embodiments 1B and 2B of the present invention;

FIG. 2D shows the results of the antibacterial activity test against P. aeruginosa in Test Example 2 of the diluted gel dressings according to Embodiments 1B and 2B of the present invention;

FIG. 3A shows the results of the cytotoxicity test on keratinocytes in Test Example 3 of the diluted gel dressing according to Embodiment 1B of the present invention;

FIG. 3B shows the results of the cytotoxicity test on THP-1 macrophages in Test Example 3 of the diluted gel dressing according to Embodiment 1B of the present invention;

FIG. 4A shows the results of the IL-1B expression test in keratinocytes in Testing example 4 of the diluted gel dressing according to Embodiment 1B of the present invention;

FIG. 4B shows the results of the IL-8 expression test in keratinocytes in Testing example 4 of the diluted gel dressing according to Embodiment 1B of the present invention;

FIG. 5A shows the results of the IL-1B expression test in keratinocytes in Testing example 4 of the diluted gel dressing according to Embodiment 1B of the present invention;

FIG. 5B shows the results of the IL-8 expression test in keratinocytes in Testing example 4 of the diluted gel dressing according to Embodiment 1B of the present invention;

FIG. 6A shows a photograph of the initial gel state of the liquid gel dressing after application according to an embodiment of the present invention;

FIG. 6B shows a photograph of the dried film state of the liquid gel dressing after application according to an embodiment of the present invention; and

FIG. 7 illustrates a photograph showing the application state of the liquid gel dressing on human skin according to Embodiment 3A of the present invention.

### DETAILED DESCRIPTION

To facilitate understanding of the present invention, some modes of implementation of the invention are described below with reference to the accompanying drawings.

### Preparation Example 1: Method of Preparing a Deep Eutectic Solvent Extract of a Chinese Medicinal Herb Composition

In this Preparation Example, referring to Table 1 below, a deep eutectic solvent extract of the Chinese medicinal herb composition in each of Embodiments 1 to 3 (i.e., E1 to E1) was prepared according to the corresponding formula in Table 1. First, Chinese medicinal herb compositions of the formulae in Table 1 were obtained, dried, ground, and then passed through a sieve of mesh size 20 to 60 to form Chinese medicinal herb composition powder (i.e., substances on which extraction was to be performed). Moreover, in this Preparation Example, betaine (Bet) was mixed with glycerol (Gly), lactate (LA), or mannose (MAN) in the mole ratios specified in Table 1 to produce the deep eutectic solvents. Once the preparation of the Chinese medicinal herb composition powder and of the deep eutectic solvents was completed, each Chinese medicinal herb composition powder was added into the corresponding deep eutectic solvent in a solid-to-liquid weight ratio of 1:10 to form a mixture. Following that, each mixture was subjected to ultrasonic extraction for 30 to 60 minutes under the following conditions: the extraction temperature being in the range from 25° C. to 35° C., the extraction power being in the range from 1000 W to 3000 W, and the ultrasonic frequency being in the range from 20 KHz to 100 kHz. After that, centrifugation was carried out at 3000 rpm for 10 minutes, followed by removal of the precipitates, filtration, and collection of the extracted liquids. The extracted liquids were then evaporated at 50° C. at such a speed that 40 wt % to 60 wt % of moisture was removed per hour, and in consequence, 100 liters of extracted liquid was reduced to 25 liters after 2 hours of condensation. Thus, each extracted liquid was reduced fourfold under a reduced pressure, and a deep eutectic solvent extract of each of the Chinese medicinal herb compositions was thereby obtained.

TABLE 1
Mole ratios of the ingredients of the deep eutectic solvents used
in Embodiments 1 to 3 (E1 to E3) to obtain deep eutectic solvent
extracts, and formulae, in weight percentage (wt %), of the substances
on which extraction was to be performed, with the symbol “—”
indicating a lack of the corresponding ingredient

| Embodiment code | | | E1 | E2 | E3 |
|---|---|---|---|---|---|
| Deep | Hydrogen | Betaine (Bet) | 1 | 1 | 1 |
| eutectic | bond acceptor | | | | |
| solvent | Hydrogen | Mannose (MAN) | 1 | — | — |
| (mole ratio) | bond donor | Lactate (LA) | — | 1 | — |
| | | Glycerol (Gly) | — | — | 2 |

| | | | | |
|---|---|---|---|---|
| Chinese medicinal herb | Artemisia argyi | 22.7 | 22.7 | — |
| composition (substance on | Houttuynia cordata | 18.2 | 18.2 | — |
| which extraction was to be | Hedyotis diffusa | 18.2 | 18.2 | — |
| performed) (wt %) | Anisomeles indica | 11.4 | 11.4 | — |
| | (L.) Kuntze | | | |
| | Lonicera japonica | 29.5 | 29.5 | — |
| | Mentha spp. | — | — | 50 |
| | Melaleuca alternifolia | — | — | 50 |

As shown in Table 1, the Chinese medicinal herb composition used in Embodiment 1 (E1) to obtain a deep eutectic solvent extract included Artemisia argyi, Houttuynia cordata, Hedyotis diffusa, Anisomeles indica (L.) Kuntze, and Lonicera japonica, and the corresponding deep eutectic solvent was a combination of betaine and mannose (in a mole ratio of 1:1). The Chinese medicinal herb composition used in Embodiment 2 (E2) to obtain a deep eutectic solvent extract included Artemisia argyi, Houttuynia cordata, Hedyotis diffusa, Anisomeles indica (L.) Kuntze, and Lonicera japonica, and the corresponding deep eutectic solvent was a combination of betaine and lactate (in a mole ratio of 1:1). The Chinese medicinal herb composition used in Embodiment 3 (E3) to obtain a deep eutectic solvent extract included Mentha spp. and Melaleuca alternifolia, and the corresponding deep eutectic solvent was a combination of betaine and glycerol (in a mole ratio of 1:2).

### Preparation Example 2: Method of Preparing a Liquid Gel Dressing

In this Preparation Example, referring to Table 2 below, each extract obtained by subjecting the Chinese medicinal herb compositions (i.e., the substances on which extraction was to be performed) in Embodiments 1 to 3 (E1 to E3) to ultrasonic extraction based on the dry weight ratios in Table 2 was added with the corresponding adjuvants according to the formulae in Table 2 to obtain the liquid gel dressings of Embodiments 1A to 3A (i.e., E1A to E3A).

TABLE 2
Formulae, in weight percentage, of the liquid gel dressings
in Embodiments 1A to 3A (E1A to E3A), with the symbol
“—” indicating a lack of the corresponding ingredient

| Embodiment code | | E1A | | E2A | | E3A | |
|---|---|---|---|---|---|---|---|
| Liquid gel | Dry weight of E1's | 15 | wt % | — | | — | |
| dressing | substance on which | | | | | | |
| | extraction was to be | | | | | | |
| | performed | | | | | | |
| | Dry weight of E2's | — | | 15 | wt % | — | |
| | substance on which | | | | | | |
| | extraction was to be | | | | | | |
| | performed | | | | | | |
| | Dry weight of E3's | — | | — | | 15 | wt % |
| | substance on which | | | | | | |
| | extraction was to be | | | | | | |
| | performed | | | | | | |
| | PVP | 8 | wt % | 8 | wt % | 8 | wt % |
| | HPMC | 2 | wt % | 2 | wt % | 2 | wt % |

| | | | | | |
|---|---|---|---|---|---|
| CaCl₂ | 0.2 | wt % | 0.2 | wt % | — |
| EDTA-2Na | 0.1 | wt % | 0.1 | wt % | — |

| | | | |
|---|---|---|---|
| Water | Remaining | Remaining | — |
| | percentage | percentage | |
| Melaleuca alternifolia | — | — | Remaining |
| distillate | | | percentage |

In Embodiment 1A (E1A), with the total weight of the liquid gel dressing constituting 100 wt %, 15 wt % of the extract of Embodiment 1 (E1) was mixed with 8 wt % of PVP, 2 wt % of HPMC, 0.2 wt % of CaCl₂), 0.1 wt % of EDTA-2Na, and water making up the remaining percentage, such that after sufficient stirring, the liquid gel dressing of Embodiment 1A (E1A) was obtained.

In Embodiment 2A (E2A), with the total weight of the liquid gel dressing constituting 100 wt %, 15 wt % of the extract of Embodiment 2 (E2) was mixed with 8 wt % of PVP, 2 wt % of HPMC, 0.2 wt % of CaCl₂), 0.1 wt % of EDTA-2Na, and water making up the remaining percentage, such that after sufficient stirring, the liquid gel dressing of Embodiment 2A (E2A) was obtained.

In Embodiment 3A (E3A), with the total weight of the liquid gel dressing constituting 100 wt %, 15 wt % of the extract of Embodiment 3 (E3) was mixed with 8 wt % of PVP, 2 wt % of HPMC, and a Melaleuca alternifolia distillate making up the remaining percentage, such that after sufficient stirring, the liquid gel dressing of Embodiment 3A (E3A) was obtained.

### Preparation Example 3: Method of Preparing a Diluted Version of a Liquid Gel Dressing

In this Preparation Example, referring to Table 3 below, the liquid gel dressings of Embodiments 1A and 2A (E1A and E2A) were used to prepare the diluted gel dressings of Embodiments 1B and 2B (i.e., E1B and E2B) according to the formulae in Table 3.

TABLE 3
Formulae, in weight percentage, of the diluted gel dressings
of Embodiments 1B and 2B (E1B and E2B), with the symbol
“—” indicating a lack of the corresponding ingredient

| Embodiment code | | E1B | E2B |
|---|---|---|---|
| Diluted gel | Liquid gel dressing of E1A | 100 wt % | — |
| dressing | Liquid gel dressing of E2A | — | 100 wt % |
| | Water (additionally added) | 60 wt % | 35 wt % |

In Embodiment 1B (E1B), with the total weight of the liquid gel dressing of Embodiment 1A (E1A) constituting 100 wt %, 60 wt % of water was additionally added to the liquid gel dressing to obtain the diluted gel dressing of Embodiment 1B (E1B).

In Embodiment 2B (E2B), with the total weight of the liquid gel dressing of Embodiment 2A (E2A) constituting 100 wt %, 35 wt % of water was additionally added to the liquid gel dressing to obtain the diluted gel dressing of Embodiment 2B (E2B).

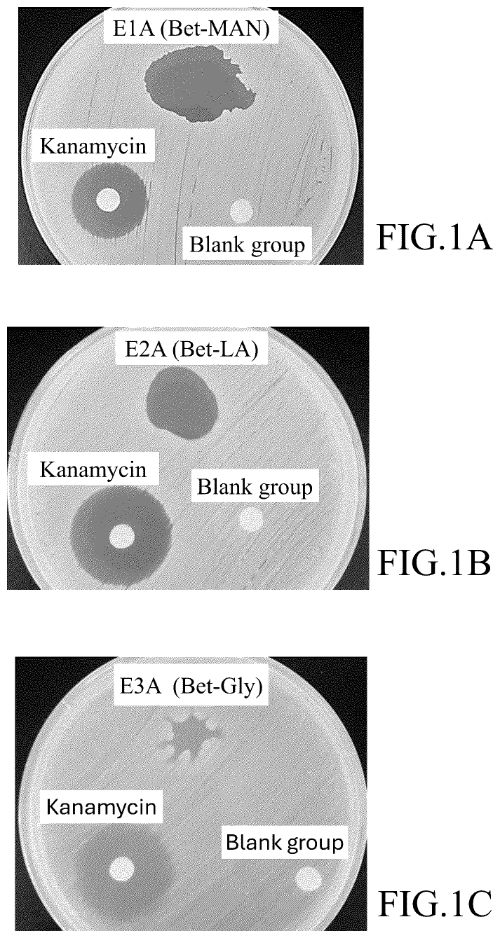

Having stated the formulae and preparation methods of the deep eutectic solvent extracts of the Chinese medicinal herb compositions in E1 to E3, of the liquid gel dressings of E1A to E3A, and of the diluted gel dressings of E1B and E2B, the present specification continues to shed light on the antibacterial and anti-inflammatory activity of the products of the foregoing embodiments by referring to FIG. 1A to FIG. 7.

### Test Example 1: Antibacterial Activity of Liquid Gel Dressings

The experimental results shown in FIG. 1A to FIG. 1C are the test results of the anti-C. acnes activity of the liquid gel dressings prepared in Embodiments 1A to 3A (E1A to E3A). This Test Example also included blank groups and kanamycin groups as the control groups.

Conducted after the liquid gel dressings of Embodiments 1A to 3A were prepared, the Test Example began by preparing culture media for C. acnes and inoculating the bacterium, which was in liquid, evenly into the surface of each culture medium to form a bacterial layer. Next, each liquid gel dressing (E1A, E2A, or E3A) was applied, in a predetermined amount and in the form of a liquid drop, to the specified surface area of one of the inoculated culture media. To set up the control groups, the same amount of deionized water was added to each culture medium to serve as a blank group, and a 20 μg/mL kanamycin solution was added to each culture medium to serve as a positive control group. The treated culture media were then placed in culture conditions suitable for growth of C. acnes (e.g., in a 37° C. anaerobic environment), allowing the bacterium to be cultured for a predetermined amount of time (about 24-48 hours). Once the culturing process was completed, the size of the inhibition zone around each sample was observed and recorded. The antibacterial activity of the liquid gel dressing of each embodiment was evaluated by comparing the sizes of the inhibition zones.

As shown in FIG. 1A to FIG. 1C, the antibacterial effects of the embodiments are as follows, in descending order: Embodiment 1A (Bet-MAN) had the highest antibacterial activity (FIG. 1A) and a better antibacterial effect than kanamycin; Embodiment 2A (Bet-LA) had the second highest antibacterial activity (FIG. 1B), which is slightly lower than that of kanamycin; and Embodiment 3A (Bet-Gly) had the lowest antibacterial activity but still produced a significant antibacterial effect (FIG. 1C). It can be known from the above that, given the same Chinese medicinal herb composition formula, the liquid gel dressing prepared with a deep eutectic solvent composed of betaine and mannose (Bet-MAN) had a superior antibacterial effect, whereas the liquid gel dressing prepared with a deep eutectic solvent composed of betaine and lactate (Bet-LA) showed a relatively low but still significant degree of antibacterial activity.

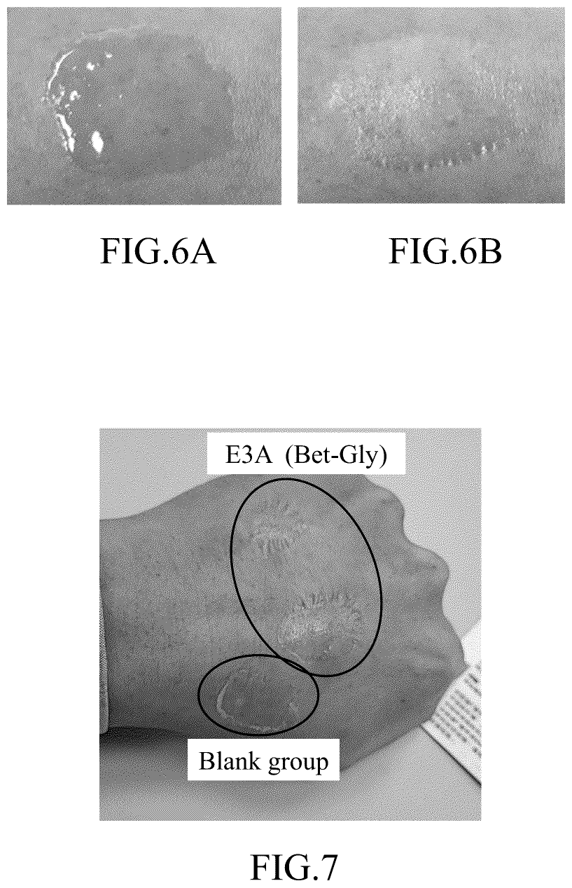

When applied to the human skin, the liquid gel dressings of Embodiments 1A to 3A were initially in a moist gel state as shown in FIG. 6A, and after about 5 to 8 seconds, each liquid gel dressing formed a dried film as shown in FIG. 6B. It is particularly worth mentioning that, referring to FIG. 7, which shows a comparison between the liquid gel dressing prepared in Embodiment 3A (E3A) and the liquid gel dressing in a blank group (which dressing had the same ingredients as in E3A, except for glycerol), the periphery of the liquid gel dressing of E3A remained tightly adhered to the skin after the dressing formed a dried film, whereas the periphery of the liquid gel dressing in the blank group peeled off the skin noticeably and formed a white edge after the dressing turned into a dried film. It can therefore be inferred that the liquid gel dressing of E3A has significantly higher affinity with the skin than the liquid gel dressing in the blank group.

### Test Example 2: Antibacterial Activity of Diluted Gel Dressings

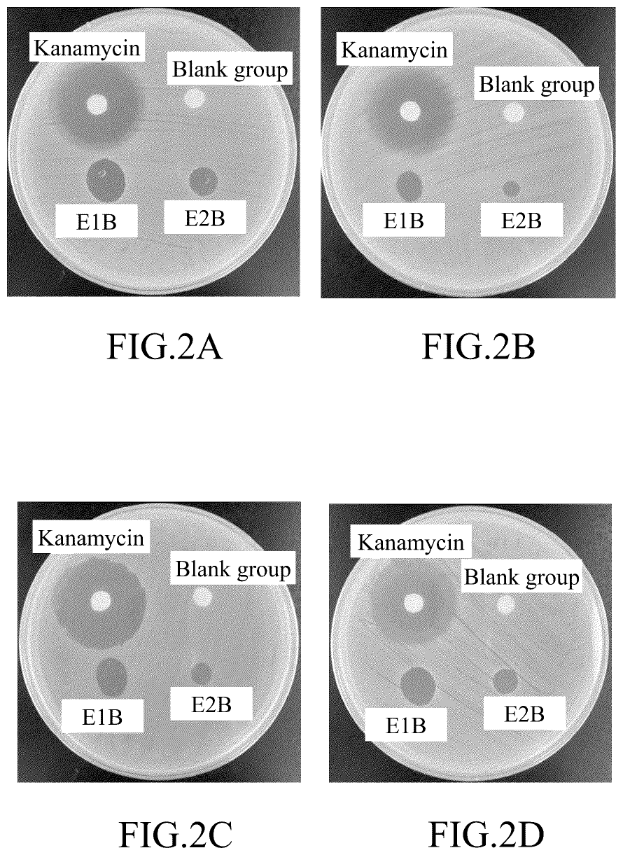

The experimental results shown in FIG. 2A to FIG. 2D are the test results of the inhibitive effects of the diluted gel dressings prepared in Embodiments 1B and 2B (E1B and E2B) on the growth activity of C. acnes (FIG. 2A), S. epidermidis (FIG. 2B), S. aureus (FIG. 2C), or P. aeruginosa (FIG. 2D). This Test Example also included blank groups and kanamycin groups as the control groups.

Conducted after the diluted gel dressings of Embodiments 1B and 2B were prepared, the Test Example began by preparing a culture medium for each of C. acnes, S. epidermidis, S. aureus, and P. aeruginosa and inoculating each bacterium, which was in liquid, evenly into the surface of the corresponding culture medium to form a bacterial layer. Next, each diluted gel dressing (E1B or E2B) was applied, in a predetermined amount and in the form of a liquid drop, to the specified surface area of each of the inoculated culture media. To set up the control groups, the same amount of deionized water was added to each culture medium to serve as a blank group, and a 20 μg/mL kanamycin solution was added to each culture medium to serve as a positive control group. After that, the treated culture media were incubated under the appropriate culture conditions for the aforementioned bacteria for a certain period of time. Once the culturing process was completed, the size of the inhibition zone around each sample was observed and recorded. The antibacterial activity of the diluted gel dressing of each embodiment was evaluated by comparing the sizes of the inhibition zones.

FIG. 2A shows the antibacterial effects on C. acnes. Embodiment 1B (E1B) formed a relatively large inhibition zone, which indicates a desirable antibacterial effect. Embodiment 2B (E2B) formed a slightly smaller inhibition zone than that of E1B but still had a certain degree of antibacterial activity. Kanamycin formed the largest inhibition zone and was therefore the most effective. The blank group did not form an inhibition zone, and this indicates a lack of antibacterial activity.

FIG. 2B shows the antibacterial effects on S. epidermidis. E1B formed a moderate-sized inhibition zone, which indicates a desirable antibacterial effect. E2B formed a slightly smaller inhibition zone than that of E1B, meaning the anti-S. epidermidis activity of E2B was slightly lower than that of E1B. Kanamycin, which served as the positive control group, formed a relatively large inhibition zone and was the most effective. The blank group did not form an inhibition zone, and this indicates a lack of antibacterial activity.

FIG. 2C shows the antibacterial effects on S. aureus. E1B formed a relatively large inhibition zone, which indicates a desirable antibacterial effect. E2B formed a slightly smaller inhibition zone than that of E1B but still showed a certain degree of antibacterial activity. Kanamycin formed the largest inhibition zone and was the most effective. The blank group did not form an inhibition zone, and this indicates a lack of antibacterial activity.

FIG. 2D shows the antibacterial effects on P. aeruginosa. E1B formed a noticeable inhibition zone but showed a slightly lower antibacterial activity than toward the other bacteria. E2B formed an even smaller inhibition zone than that of E1B, and this means that E2B had the weakest antibacterial effect on P. aeruginosa of all the bacteria. Kanamycin still formed the largest inhibition zone and therefore had the most significant antibacterial effect. The blank group did not form an inhibition zone, and this indicates a lack of antibacterial activity.

It can be known from the above that, given the same Chinese medicinal herb composition formula, the diluted gel dressing prepared with a deep eutectic solvent composed of betaine and mannose (Bet-MAN) had a superior antibacterial effect, whereas the diluted gel dressing prepared with a deep eutectic solvent composed of betaine and lactate (Bet-LA) showed a relatively low but still significant degree of antibacterial activity.

### Test Example 3: Evaluation of the Cytotoxicity of Diluted Gel Dressings

This Test Example was intended to evaluate the effects of different concentrations of the Chinese-medicinal-herb-composition deep eutectic solvent extract in the diluted gel dressing of Embodiment 1B (E1B) on the cell survival rates of HaCaT cells (a keratinocyte cell line) and THP-1 macrophages, thereby determining whether or not the deep eutectic solvent extract is cytotoxic.

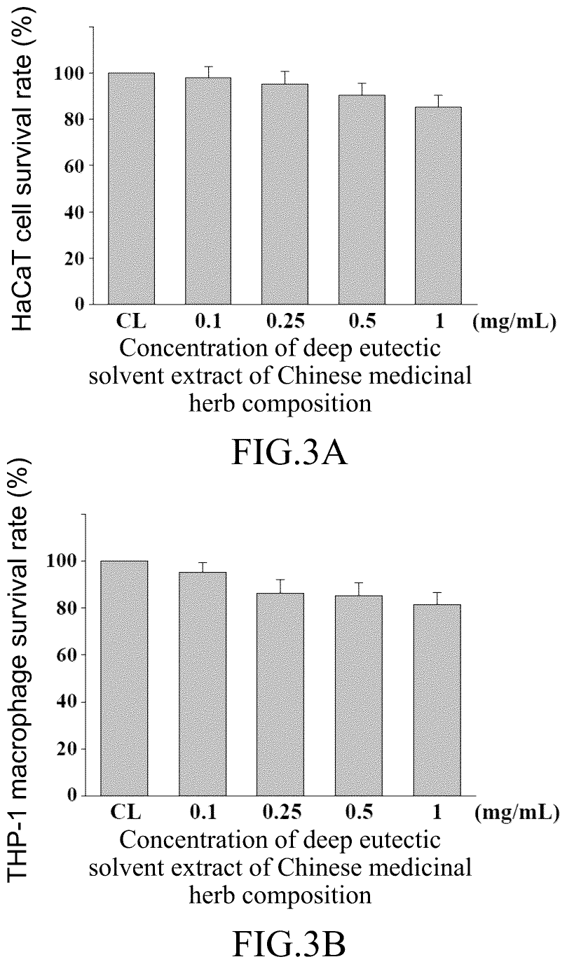

FIG. 3A shows the effects on HaCaT cells. The diluted gel dressing of Embodiment 1B (E1B) was diluted in such a way that the concentration of the deep eutectic solvent extract of the Chinese medicinal herb composition was reduced from 100 mg/mL to 0.1-1 mg/mL, and each of the resulting diluted gel dressings was added to a HaCaT cell culture medium. As can be seen in FIG. 3A, the HaCaT cell survival rates were all higher than 90%. Although the cell survival rates lowered slightly when the concentration was increased (to 0.25 mg/mL through 1 mg/mL), the differences in cell survival rate were not significant. Therefore, it can be inferred that even if the diluted gel dressing of Embodiment 1B (E1B) has a concentration as high as 1 mg/ml, the Chinese-medicinal-herb-composition deep eutectic solvent extract in the dressing still has very low cytotoxicity toward HaCaT cells. This means that the diluted gel dressing is safe and is suitable for use in skincare products.

FIG. 3B shows the effects on THP-1 macrophages. As can be seen in FIG. 3B, the macrophage survival rates stayed between 85% and 90% when the concentration of the Chinese-medicinal-herb-composition deep eutectic solvent extract in the diluted gel dressing of Embodiment 1B (E1B) was in the range of 0.1-1 mg/mL. Although the macrophage survival rate at the highest concentration, i.e., 1 mg/mL, was slightly lower than the corresponding HaCaT cell survival rate, the former nevertheless remained at a relatively high level. It can therefore be inferred that as far as macrophages are concerned, the Chinese-medicinal-herb-composition deep eutectic solvent extract in the diluted gel dressing of Embodiment 1B (E1B) does not have significant toxicity when its concentration is within the test range, and this indicates that the composition is suitable for use in anti-inflammatory drugs or preparations for external use.

It can be known from the foregoing results that the cell survival rates lowered slightly as the concentration rose, and that the overall changes in survival rate, however, were not significant, meaning the cytotoxicity of the extract is not significantly related to the concentration of the extract. The experimental results also show that the Chinese-medicinal-herb-composition deep eutectic solvent extract in Embodiment 1B allowed both the HaCaT cells and the macrophages to have high survival rates. Thus, it is verified that the extract is safe, has great application potential, and is particularly suitable for use in anti-inflammation-related skincare products or immune-related treatments. The results provide sufficient support for using the extract as a low-toxicity and high-biocompatibility candidate material in subsequent clinical and product development.

### Test Example 4: Evaluation of the Anti-Inflammatory Effects of Diluted Gel Dressings

The diluted gel dressing of Embodiment 1B (E1B) showed a desirable inhibitive effect on C. acnes-induced expression of the IL-1B and IL-8 pro-inflammatory genes in keratinocytes (HaCaT cells) and THP-1 macrophages.

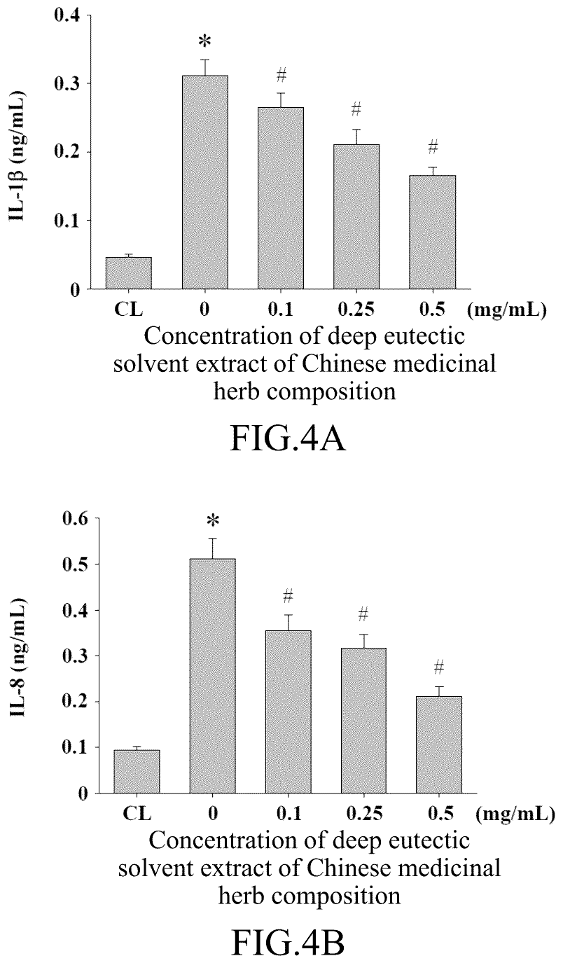

It can be seen in FIG. 4A that when the diluted gel dressing was not added (i.e., when the concentration was 0 mg/mL), the IL-1B expression in the keratinocytes (HaCaT cells) increased significantly; that the IL-1B expression was gradually reduced as the concentration of the dressing rose to 0.1 mg/mL, 0.25 mg/mL, and 0.5 mg/ml; and that a significant inhibitive effect was achieved when the concentration was 0.5 mg/mL. As to the expression of IL-8, a similar trend can be seen in FIG. 4B: the IL-8 expression increased significantly in the absence of the diluted gel dressing, was gradually reduced when the concentration of the dressing was increased, and was significantly inhibited when the concentration was the highest. The results show that the Chinese-medicinal-herb-composition deep eutectic solvent extract in the diluted gel dressing of Embodiment 1B was effective in inhibiting C. acnes-induced inflammatory response and had a desirable concentration-dependent inhibitive effect on the expression of the IL-1B and IL-8 genes in the keratinocytes (HaCaT cells) and the macrophages, and this proves that the extract is anti-inflammatory and has the potential of being used in skincare products.

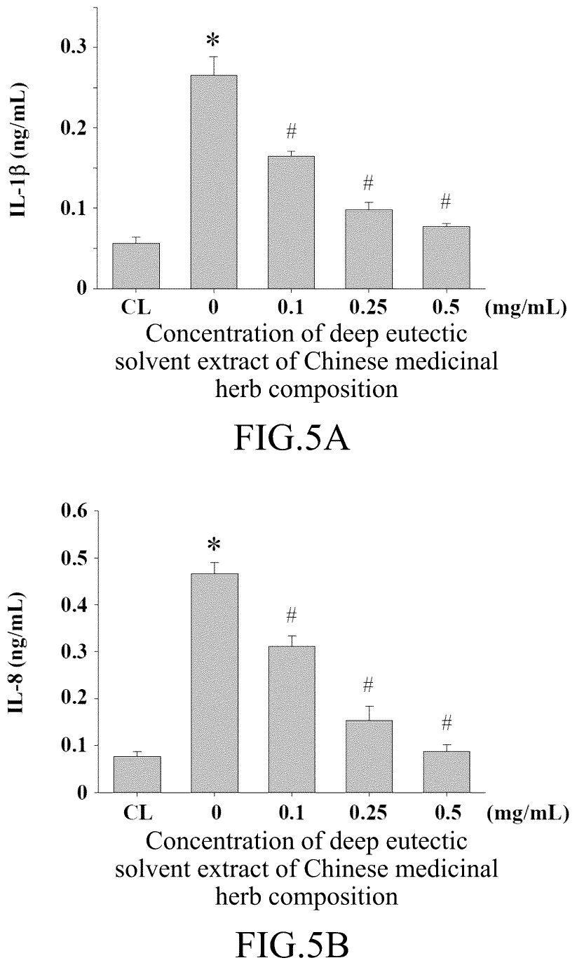

It can be seen in FIG. 5A that when the diluted gel dressing was not added (i.e., when the concentration was 0 mg/mL), the IL-1B expression in the THP-1 macrophages increased significantly; that the IL-1B expression was gradually reduced as the concentration of the diluted gel dressing rose to 0.1 mg/mL, 0.25 mg/mL, and 0.5 mg/mL; and that the strongest inhibitive effect was achieved when the concentration was 0.5 mg/mL. As to the expression of IL-8, a similar trend can be seen in FIG. 5B: the IL-8 expression increased significantly in the absence of the diluted gel dressing, was noticeably reduced when the concentration of the diluted gel dressing was increased, and was significantly inhibited when the concentration reached 0.5 mg/mL. The results show that the Chinese-medicinal-herb-composition deep eutectic solvent extract in the diluted gel dressing of Embodiment 1B was effective in inhibiting C. acnes-induced inflammatory response in the THP-1 macrophages and had a concentration-dependent inhibitive effect on the expression of IL-1B and IL-8 genes, and this further proves that the extract has desirable anti-inflammatory performance and the potential of being used in anti-inflammatory treatments and related products.

According to the above, the present invention uses a deep eutectic solvent to extract the active ingredients of a Chinese medicinal herb composition, wherein the deep eutectic solvent is advantageous in that it has low volatility, is non-flammable, can dissolve various plant-based compounds, is chemically stable, can be adjusted in viscosity, and can be prepared rapidly. Moreover, the strong hydrogen bonds between the ingredients of the deep eutectic solvent and the extract help increase the yield of the extract. The invention uses betaine in combination with mannose, lactate, or glycerol as the deep eutectic solvent, and it has been proved that the deep eutectic solvent extract obtained from the Chinese medicinal herb composition by ultrasonic extraction with the deep eutectic solvent has a significant antibacterial and inflammation-inhibiting effect. In addition, the use of ultrasonic extraction not only makes it possible to obtain the deep eutectic solvent extract of the Chinese medicinal herb composition rapidly through a simple process, but also allows the extraction performed on the Chinese medicinal herb composition to be non-high-temperature extraction and be completed within a short time to preserve the thermosensitive plant-based chemical substances. Therefore, the Chinese-medicinal-herb-composition deep eutectic solvent extract of the invention can be used to prepare the pharmaceutical compounds in liquid gel dressings while meeting the requirements of green chemistry.

What is claimed is:

1. A use of a deep eutectic solvent extract of a Chinese medicinal herb composition in preparing a pharmaceutical composition with antibacterial and anti-inflammatory activity, wherein the deep eutectic solvent extract is obtained by performing ultrasonic extraction on the Chinese medicinal herb composition through a deep eutectic solvent and by performing a concentration process after the ultrasonic extraction, the deep eutectic solvent includes betaine (Bet) as a hydrogen bond acceptor (HBA), the deep eutectic solvent further includes a hydrogen bond donor (HBD) selected from the group consisting of glycerol (Gly), lactate (LA), and mannose (MAN), and the Chinese medicinal herb composition is selected from the group consisting of a combination of Artemisia argyi, Houttuynia cordata, Hedyotis diffusa, Anisomeles indica (L.) Kuntze, and Lonicera japonica and a combination of Mentha spp, and Melaleuca alternifolia.

2. The use of claim 1, wherein the deep eutectic solvent is prepared by mixing betaine and glycerol in a mole ratio in a range from 1:1.5 to 1:2.5.

3. The use of claim 1, wherein the deep eutectic solvent is prepared by mixing betaine and lactate in a mole ratio in a range from 1:0.5 to 1:1.5.

4. The use of claim 1, wherein the deep eutectic solvent is prepared by mixing betaine and mannose in a mole ratio in a range from 1:0.5 to 1:1.5.

5. The use of claim 1, wherein with a total weight of the Chinese medicinal herb composition constituting 100 wt %, the Chinese medicinal herb composition comprises 20 wt % to 25 wt % of Artemisia argyi, 15 wt % to 20 wt % of Houttuynia cordata, 15 wt % to 20 wt % of Hedyotis diffusa, 9 wt % to 13 wt % of Anisomeles indica (L.) Kuntze, and 27 wt % to 32 wt % of Lonicera japonica.

6. The use of claim 5, wherein the pharmaceutical composition comprises a liquid gel dressing, and with the liquid gel dressing constituting 100 wt %, the liquid gel dressing comprises a said deep eutectic solvent extract obtained from a said Chinese medicinal herb composition whose dry weight constitutes 10 wt % to 20 wt % of the liquid gel dressing, 5 wt % to 11 wt % of polyvinyl pyrrolidone (PVP), 1 wt % to 3 wt % of hydroxypropyl methylcellulose (HPMC), 0.1 wt % to 0.3 wt % of calcium chloride (CaCl₂)), 0.05 wt % to 0.15 wt % of disodium ethylenediaminetetraacetate (EDTA-2Na), and water making up a remaining percentage of the liquid gel dressing.

7. The use of claim 6, wherein the pharmaceutical composition comprises the liquid gel dressing and water additionally added to the liquid gel dressing, and with the liquid gel dressing constituting 100 wt %, the additionally added water constitutes 55 wt % to 65 wt %.

8. The use of claim 1, wherein with a total weight of the Chinese medicinal herb composition constituting 100 wt %, the Chinese medicinal herb composition comprises 45 wt % to 55 wt % of Mentha spp, and 45 wt % to 55 wt % of Melaleuca alternifolia.

9. The use of claim 8, wherein the pharmaceutical composition comprises a liquid gel dressing, and with the liquid gel dressing constituting 100 wt %, the liquid gel dressing comprises a said deep eutectic solvent extract obtained from a said Chinese medicinal herb composition whose dry weight constitutes 10 wt % to 20 wt % of the liquid gel dressing, 5 wt % to 11 wt % of polyvinyl pyrrolidone (PVP), 1 wt % to 3 wt % of hydroxypropyl methylcellulose (HPMC), and a Melaleuca alternifolia distillate making up a remaining percentage of the liquid gel dressing.

10. The use of claim 1, wherein the Chinese medicinal herb composition is Chinese medicinal herb composition powder having been dried, crushed, and passed through a sieve of mesh size 20 to 60, and a solid-to-liquid weight ratio between the Chinese medicinal herb composition and the deep eutectic solvent is in a range from 1:5 to 1:15.

11. The use of claim 1, wherein the pharmaceutical composition is a liquid gel dressing for inhibiting growth activity of Cutibacterium acnes (C. acnes).

12. The use of claim 5, wherein the pharmaceutical composition is a liquid gel dressing for inhibiting growth activity of Cutibacterium acnes (C. acnes).

13. The use of claim 6, wherein the pharmaceutical composition is the liquid gel dressing and is used to inhibit growth activity of Cutibacterium acnes (C. acnes).

14. The use of claim 8, wherein the pharmaceutical composition is a liquid gel dressing for inhibiting growth activity of Cutibacterium acnes (C. acnes).

15. The use of claim 9, wherein the pharmaceutical composition is the liquid gel dressing and is used to inhibit growth activity of Cutibacterium acnes (C. acnes).

16. The use of claim 11, wherein when the deep eutectic solvent is a combination of betaine and mannose (Bet-MAN), water constituting 55 wt % to 65 wt % of the liquid gel dressing is additionally added to the liquid gel dressing to form a first diluted gel, and the first diluted gel is used to inhibit growth activity of C. acnes, Staphylococcus epidermidis (S. epidermidis), Staphylococcus aureus (S. aureus), or Pseudomonas aeruginosa (P. aeruginosa).

17. The use of claim 16, wherein the first diluted gel is used to inhibit inflammation and has no cytotoxicity toward keratinocytes or THP-1 macrophages.

18. The use of claim 16, wherein the first diluted gel is used to inhibit C. acnes-induced IL-1B expression and C. acnes-induced IL-8 expression in HaCaT cells.

19. The use of claim 12, wherein the first diluted gel is used to inhibit C. acnes-induced IL-1B expression and C. acnes-induced IL-8 expression in THP-1 macrophages.

20. The use of claim 11, wherein when the deep eutectic solvent is a combination of betaine and lactate (Bet-LA), water constituting 30 wt % to 40 wt % of the liquid gel dressing is additionally added to the liquid gel dressing to form a second diluted gel, and the second diluted gel is used to inhibit growth activity of C. acnes, Staphylococcus epidermidis (S. epidermidis), Staphylococcus aureus (S. aureus), or Pseudomonas aeruginosa (P. aeruginosa).
